(19) United States
(12) Patent Application Publication
(10) Pub. No.: US 2008/0269853 A1
(43) Pub. Date: Oct. 30, 2008

(54) Thermotherapy Device

(75) Inventor: Katsumi Kitanaka, Tokyo (JP)

Correspondence Address: OLIFF & BERRIDGE, PLC, P.O. BOX 320850, ALEXANDRIA, VA, 22320-4850, US

(73) Assignee: MICHIKO KITANAKA, Tokyo (JP)

(21) Appl. No.: 11/628,197

(22) PCT Filed: Jun. 3, 2004
(86) PCT No.: PCT/JP04/07712

§ 371 Date: Jan. 18, 2007

(51) Int. Cl. A61F 7/00

(52) U.S. Cl. 607/108

(57) ABSTRACT

A thermotherapy device comprises a cup-like housing (1) having a heat radiation opening (1a), a rod-like electric heater (2) contained in the housing (1) with the leading end thereof being directed toward the heat radiation opening (1a), and a controller (3) for supplying power to the electric heater (2) and controlling the amount of heat generation from the electric heater (2). Since the electric heater (2) is employed, replacement of a combustion rod and handling of combustion ash are not required, and the thermotherapy device can be used even in bed. Moreover, power is supplied to the electric heater (2) while regulating the amount of heat generation by means of controller (3). Accordingly, the skin (S) can be heated in safety to a high temperature of 55 to 60 degrees C., and an affected part (A) located on or under the skin (S) can be exposed to a sufficiently high temperature for treatment of cancer.

### FIELD OF THE INVENTION

The present invention relates to a thermotherapy device for an effective treatment of diseases including cancer, by heating an affected part on the skin or inside the body under the skin.

### BACKGROUND OF THE INVENTION

As a device for a treatment of a disease by heating an affected part on or under the skin is known, for example, a heat supply device comprising as a heat source a combustion rod (see Japanese, Utility Model Application Laid-Open No. S63-103618).

This heat supply device comprises a combustion rod composed of moxa caked into the shape of a rod and supported inside a support tube. The air inside the support tube is warmed up by combustion at the tip of the combustion rod to become a warm air and to be blown onto the skin around the affected part by an electric fan.

However, as for this heat supply device, troublesome replacement of the combustion rod and handling of combustion ash are needed as the combustion rod is consumed. Further, there is a danger of burn injury or fire disaster if the device is used, for example, in bed or blanket. In addition, the electric fan being large and heavy, it is difficult to expose for a long time the skin around the affected part to a warm air if the device is carried in hand.

A warming device comprising as a heat source a ceramic heater, a kind of electric heaters, is known which is intended to overcome the disadvantages described above (see Japanese Patent Application Laid-Open No. 2001-276236).

This warming device comprises a disk-like pad having a diameter of about 4 cm, including a cover in the shape of a thick disk having an opening on the fore side and a ceramic heater in the shape of a circular plate placed in the cover toward the opening and a battery equipment for supplying power to the ceramic heater through a cable. The pad is fixed on the skin around the affected part with an adhesive tape or a band, and the ceramic heater is then supplied with a certain amount of electrical power to warm up the affected part.

As for this warming device, however, since the opening is as small as about 2 cm in diameter because the diameter of the pad is made small for the purpose of portability, the heat concentrates on a single point on the skin. Further, the device is not provided with a function of controlling the amount heat generation. Thus, for safety reasons, treatment is only possible to such an extent as to slightly warm up the ski, and there is difficulty in providing a treatment with a high temperature sufficient and effective for e.g. treatment of cancer.

### DISCLOSURE OF THE INVENTION

### Problems to be Solved

The present invention, therefore, is intended to provide a thermotherapy device that does not require replacement of a combustion rod or handling of combustion ash, and can be held by hand, and makes possible a safe thermotherapy with a sufficiently high temperature.

### Means for Solving the Problems

The thermotherapy device of the present invention, for advantageously solving the problems, comprises a cup-like housing having a heat radiation opening, a rod-like electric heater contained in said housing with the leading end thereof being directed toward said heat radiation opening and a controller for supplying power to said electric heater and controlling the amount of heat generation from the electric heater.

With a thermotherapy device having such a configuration of the present invention, by holding the housing with hand or with an equipment to be described hereinafter in such a way that the heat radiation opening of the housing is directed toward the skin around the affected part and by supplying power to the electric heater with the use of the controller, whole of the rod-like electric heater with the leading end thereof being directed toward the heat radiation opening, warms up the air inside the cup-like housing to make convection of the air, and the heat is transmitted onto the skin by convection and also by radiation from the leading end of the electric heater. The affected part on the skin or inside the body under the skin can thus be heated continuously.

Further, with the thermotherapy device of the present invention, due to the electric heater being used, there is no problem of replacement of a combustion rod or handling of combustion ash, which makes it possible to use the device at any time, even in bed or blanket. In addition, since the electric heater is supplied with power while regulating the amount of heat generation by means of the controller, the skin may be heated up safely to a high temperatures in the range of 55 to 60 degrees C. to heat up the affected part on or under the skin to such a high temperature as is sufficient and effective for e.g. cancer treatment.

According to a test of the device using the inventor's own body as a test subject, in a case that a heat around 40 degrees C. was applied onto the skin, there was rather an internal feeling of worsening of a prostatic cancer as an affected part. In another case, on the other hand, that a heat around 55 to 60 degrees C. was applied onto the skin, there was a feeling of kind of piping hot blood and a spark of life evoked into the muscle. Finally, the prostatic cancer of the inventor's own body was completely cured by repeating application of heat of a high temperature, as will be described hereinafter.

As for the thermotherapy device of the present invention, the diameter of the heat radiation opening in the above-described housing is preferably in the range of 4 to 6 cm, and the leading end of the above-described electric heater is preferably located 1 to 3 cm in the back of the heat radiation opening. With this, excessive concentration and diffusion of heat from the electric heater can be avoided due to the heat radiation opening having a diameter in the range of 4 to 6 cm. Further, due to the rod-like electric heater having the leading end thereof located 1 to 3 cm in the back of the heat radiation opening, the air inside the cup-like housing is warmed up in whole to transmit the heat onto the skin by convection as well as by infrared radiation from the leading end thereof. Thus, the affected part on or under the skin can be heated up to a sufficiently high temperature more safely.

It is also preferable to provide the thermotherapy device of the present invention with a heat insulation material between the above-described housing and the above-described electric heater. With this, increase in temperature of the housing due to the heat generated by the electric heater can be limited, and a burden on the patient can be alleviated when the housing is held by hand.

Further, it is preferable to provide the thermotherapy device of the present invention with a reflector between the above-described housing and the above descried electric heater. With this, increase in temperature of the housing due to the heat generated from the electric heater can be inhibited, alleviating a burden on the patient when the housing is carried in hand, and efficiency in warming up the air inside the housing and infrared radiation onto the skin is improved.

Although the above-described housing of the thermotherapy device of the present invention may be made of plastic, more preferably, the housing is made of wood or ceramics. Wood and ceramics having much lower thermal conductivity than metal, increase in temperature of the housing due to the heat generated from the electric heater can be inhibited if the housing is made of wood or ceramics, alleviating a burden on the patient when the housing is carried in hand.

Further, it is preferable to provide the thermotherapy device of the present invention with a net member on the inner side of the above-described opening for radiation of the above-described housing. With this net member, even when treatment is done in bed or blanket or when the housing is pressed against the skin using a towel, it is possible to securely prevent such a cloth or the tip of a finger from slipping into the housing from the heat radiation opening by accident.

Further, the thermotherapy device of the present invention is preferably provided with a temperature sensor near the heat radiation opening, and the above-described controller preferably controls the amount of heat generation from the above-described electric heater, based on an output signal from the temperature sensor. With this, since the controller, without being operated by hand, [symbol] temperature during thermotherapy can be eliminated, allowing the patient to sleep during thermotherapy. The above-described controller may be provided with a timer for setting a start time, an ending time and/or a period of time of power supply, etc.

Further, the above-described controller of the thermotherapy device of the present invention is preferably provided with a temperature adjustment knob for changing the amount of heat generation from the above-described electric heater and a display area where a correspondence between a turn position of the temperature adjustment knob and a target temperature is indicated using different colors. As an example, by representing the temperature range (or approximate temperature) of 40 to 45 degrees C. by white, the temperature range of 45 to 50 degrees C. by green, the temperature range of 50 to 55 degrees C. by yellow and the temperature range of 55 to 60 degrees C. by red, it is possible to set the controller so as to heat up the skin to a desired temperature only by turning the temperature adjustment knob according to the color-coded display. The operation of the device may thus be made extremely easy.

The thermotherapy device of the present invention is preferably provided with a mounting fixture, such as a belt having a hook and loop fastener for adjusting the length or changing the form, for fixing the heat radiation opening of the housing around the affected part. In this manner, there being no need for holding the housing by hand during thermotherapy, the burden on the patient can further be alleviated, and the housing can always be fixed on an accurate position around the affected part each time of repeated thermotherapeutic treatments. Effectiveness of treatment can thus be improved.

### BRIEF DESCRIPTION OF THE DRAWINGS

FIG. 1 is a cross-sectional view of a thermotherapy device according to an embodiment of the present invention, in which the device is used in such a way as to be attached firmly onto the skin around an affected part.

FIG. 2 is a block diagram showing a configuration of a controller of the thermotherapy device of the above-described embodiment.

FIG. 3 is a schematic diagram showing how to use the thermotherapy device of the above-described embodiment.

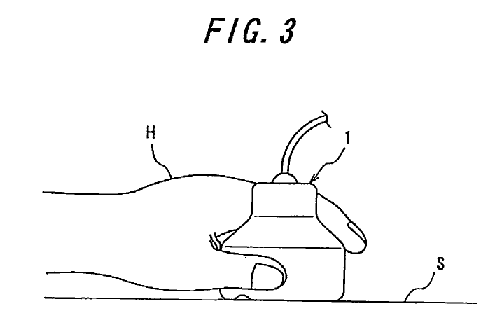

FIG. 4 is a characteristic line diagram showing a result of a test of the thermotherapy device of another embodiment of the present invention, in which the inventor's own body was used as a test subject, and a more simplified thermotherapy device was used as the embodiment.

FIG. 5 is a plan view on a thermotherapy device according to a third embodiment of the present invention.

### BEST MODES FOR CARRYING OUT THE INVENTION

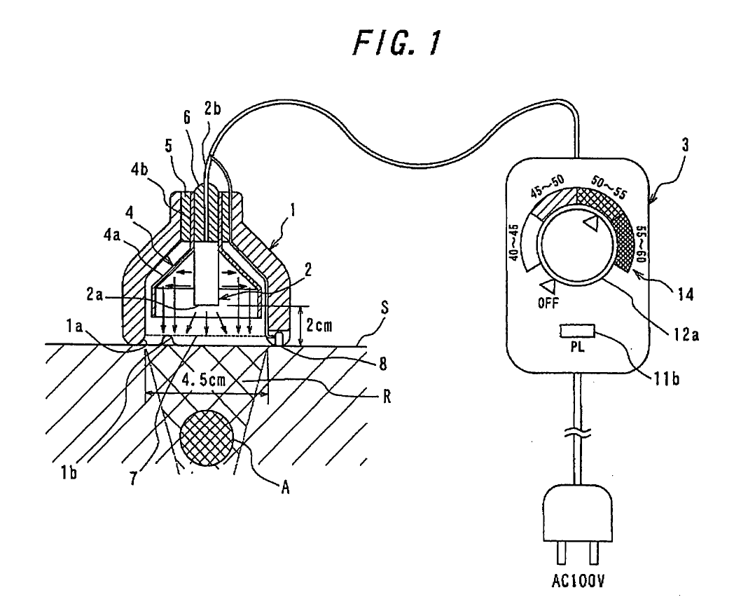

The preferred embodiments of the present invention will described hereinafter with reference to figures. FIG. 1 is a cross-sectional view showing an embodiment of the thermotherapy device of the present invention, in which the device is being used in such a way as to be attached firmly onto the skin around the affected part. The thermotherapy device according to the present embodiment comprises a cup-like housing 1 having at the fore thereof (at the bottom in FIG. 1) a heat radiation opening 1a, a cartridge heater 2 having an approximate diameter of 9 mm and an approximate length of 27 mm (for example, a FIREROD manufactured by Watlow Japan Co., Ltd.) as a rod-like electric heater contained in the housing in such a way that the leading end 2a thereof is directed toward the heat radiation opening 1a and a controller 3 for supplying power to the cartridge heater 2 and controlling the amount of heat generation from the cartridge heater 2.

The housing 1 is made of wood (white oak, for example), a material of relatively high performance in heat insulation. The diameter of the heat radiation opening 1a of the housing 1 is in the range of 4 to 6 cm, and more preferably about 4.5 cm. The cartridge heater 2 is located in the center of the housing 1 with the leading end thereof 2a being located 1 to 3 cm, more preferably about 2 cm, in the back of the heat radiation opening 1a.

Inside the housing 1 is provided a reflector 4 having a reflecting part 4a in the shape of a circular cone without its top and a supporting part 4b in the shape of a circular cylinder in such a way that a clearance gap is formed between the reflecting part 4a and the inner wall of the housing 1. The supporting part 4b of the reflector 4 is supported at the rear end (on top in FIG. 1) of the housing 1, and a heat insulation material 5 (a ceramic fiber, for example) in the shape of a circular cylinder is provided between the supporting part 4b and the rear end of the housing 1.

The rear end of the cartridge heater 2 is supported so as to be fitted in the supporting part 4b of the reflector 4. Inside the supporting part 4b of the reflector 4 is filled up a heat resistance resin 6 for fixing a power supply wire 2b passing through the supporting part 4b.

On the inner side of the heat radiation opening 1a of the housing 1 is further provided a net member 7 (a net made of a carbon fiber, for example) for preventing a finger or a cloth from slipping into the housing 1. At the front end of the housing 1 is formed a plurality of recesses for air flow 1b at positions equally spaced along the circumferential direction. On the inside of one of the recesses for air flow 1b is provided a temperature sensor (a thermistor, for example) in such a way that the sensor may contact the skin S when the front end of the housing 1 is pressed against the skin S.

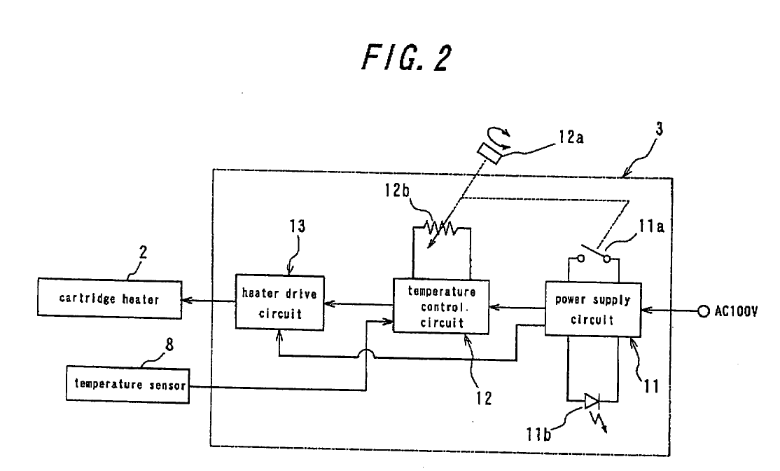

The controller 3 as shown in FIG. 2 comprises:

a power supply circuit 11 having an on/off switch 11a and a pilot lamp 11b for adjusting a commercial power source of AC 100 V to such an electrical power as is suitable for the cartridge heater 2,

a temperature control circuit (for example, an analog circuit or a microcomputer) having a variable resistor 12b for changing the value of resistance according to a turning operation of the temperature adjustment knob 12a shown in FIG. 1 in conjunction with the on/off switch 11a, which is connected to a temperature sensor 8 and outputs a control signal generated by adjusting an electrical power supplied from the power supply circuit 11 based on the value of resistance of the variable resistor 12b and that of the temperature sensor 8, and

a heater drive circuit 13 (for example, a combinational circuit of a known triac and a diac) for controlling the amount of heat generation from the cartridge heater 2 by adjusting the electrical power supplied from the power supply circuit 11 using the control signal (a control current for example) output from the temperature control circuit 12, the electrical power then being supplied to the cartridge heater 2.

The controller 3 is provided with a display area 14 where correspondence between a turn position of the temperature adjustment knob 12a and a target temperature of heat applied onto the skin from the cartridge heater 2, as shown in FIG. 1. The display area 14 is separated by color according to different temperature ranges, based on results of an experiment in advance. For example, the temperature range of 40 to 45 degrees C. is represented by white, and the temperature range of 45 to 50 degrees C. is represented by green, the temperature range of 50 to 55 degrees C. is represented by yellow, and the temperature range of 55 to 60 degrees C. is represented by red. The temperature of the cartridge heater 2 is about 160 to 180 degrees C. when the temperature of the skin S is 50 to 60 degrees C., according to an experiment that was carried out by the inventor of the present invention.

When using the thermotherapy device of the present embodiment, the housing 1 is held with hand by the patient or nurse, and the heat radiation opening 1a is pressed against the skin S around the affected part. The on/off switch 11a is turned on firstly, and the temperature adjustment knob 12a is then turned in such a way that a triangle-shaped symbol on the temperature adjustment knob 12a is directed toward a target temperature. With this, the controller 3 controls a current supplied to the cartridge heater 3, with the use of data of temperature of the inside of the housing 1 near the skin S having been fed back from the temperature sensor 8, so that the temperature of the skin S is in the range of the target temperature set with the temperature adjustment knob 12a.

Accordingly, with the use of the thermotherapy device of the present embodiment, the cartridge heater 2 as a whole, with the leading end thereof 2a being directed toward the heat radiation opening 1a, warms up the air inside the cup-like housing 1, and the heat is transmitted onto the skin S by convection of the air as well as by infrared radiation from the leading end 2a. Thus, a region R thoroughly including the affected part A located on the skin S or inside the body under the skin S can continuously be heated.

Further, with the use of the thermotherapy device of the present embodiment, there is no need for replacement of a combustion rod or handling of combustion ash because the cartridge heater 2 is used. Thus, it is possible to use the device even in bed. What is more, because the cartridge heater 2 is supplied with power while regulating the amount of heat generation by means of the controller 3, the skin S can be safely heated to a high temperature of 55 to 60 degrees C. The affected part A located on or under the skin S can thus be heated to a sufficiently high temperature suitable for treatment of diseases including cancer.

Further, with the use of the thermotherapy device of the present embodiment, the heat radiation opening 1a having a diameter of 4 to 6 cm, and more preferably about 4.5 cm, excessive concentration or diffusion of heat generated from the cartridge heater 2 is prevented. The cartridge heater 2, the leading end thereof being located 1 to 3 cm, more preferably about 2 cm, in the back of the heat radiation opening 1a, warms up the air inside the cup-like hosing in whole, and the heat is transmitted onto the skin by convection of the air as well as infrared radiation from the leading end 2a. Thus, a large region R, not only the surface of the skin S but also some centimeters backward of the skin, can be exposed to a high temperature, although the region is narrowing toward the back. With this, the affected part A located even some centimeters under the skin S can wholly be heated to a sufficiently high temperature safely.

Further, with the use of the thermotherapy device of the present embodiment, due to the heat insulation material 5 provided between the housing 1 and the cartridge heater 2, increase in temperature of the housing 1 caused by the heat from the cartridge heater 2 is inhibited. This may alleviate a burden on the patient or nurse when the housing is held by hand.

Further, with the use of the thermotherapy device of the present embodiment, due to the reflector 4 provided between the housing 1 and the cartridge heater 2, increase in temperature of the housing 1 caused by the heat from the cartridge heater 2 is inhibited to alleviate a burden on the patient or nurse when the housing is held by hand, and efficiency in warming up the air inside the housing 1 and infrared radiation onto the skin S may be increased because the whole infrared radiation from the cartridge heater 2 can be directed toward the heat radiation opening 1a, as shown with arrows in FIG. 1.

Further, with the use of the thermotherapy device of the present embodiment, because the housing 1 is made of wood, a material generally having a much lower heat conductivity compared with that of metal, increase in temperature of the housing 1 caused by Me heat from the cartridge heater 2 is inhibited to alleviate a burden on the patient or nurse when the housing is held by hand.

Further, with the use of the thermotherapy device of the present embodiment, due to the net member 7 provided on the inner side of the heat radiation opening 1a of the housing 1a, it is possible to securely prevent a cloth or the tip of a finger from accidentally slipping into the housing through the heat radiation opening 1a, when treatment is done in bed, or the housing 1 is pressed against the skin S using a towel.

Further, with the use of the thermotherapy device of the present embodiment, due to the temperature sensor 8 provided near the heat radiation opening 1a of the housing 1 and the controller 3 that controls the amount of heat generation from the cartridge heater 2 based on an output signal from the temperature sensor 8, the amount of heat generation from the cartridge heater 2 is automatically controlled by the controller 3 to an appropriate extent, without manual operation of the temperature adjustment knob 12a, based on data of temperature at a position close to the skin S. Thus, the need for adjusting temperature during thermotherapy can be eliminated, allowing the patient to sleep during thermotherapy.

Further, with the use of the thermotherapy device of the present embodiment due to the controller 3 provided with the temperature adjustment knob 12a for changing the amount of heat generation from the cartridge heater 2 and the display area 14 in which correspondence between a turn position of the temperature adjustment knob 12a and a target heating temperature is shown by different colors, for example, white for 40 to 45 degrees C., green for 45 to 50 degrees C., yellow for 50 to 55 degrees C. and red for 55 to 60 degrees C., the controller 3 can be set so as to heat the skin S to a desired temperature only by turning the temperature adjustment knob 12 a according to the different colors. Thus, the operation of the device can be made extremely easy.

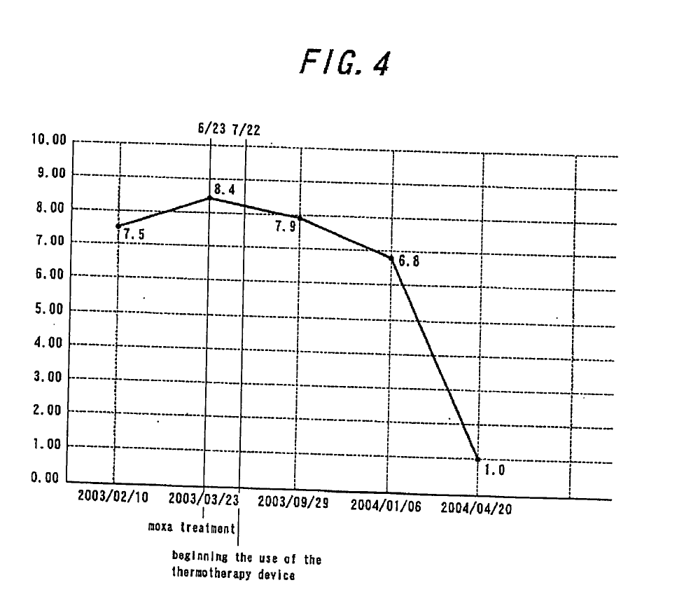

FIG. 4 is a characteristic line diagram showing a result of an experiment that was carried out by the inventor of the present invention, in which the thermotherapy device according to another embodiment of the present invention was used. In this embodiment, the thermotherapy device of the above-described embodiment is simplified in such a way that the reflector 4 and the temperature sensor 8 are removed. In other words, there is no feedback of temperature data. Further, a commercial dimmer (for example, one named Lucon TMC-201W manufactured by Toyostar Co., Ltd.) is used as a controller 3.

The inventor of the present invention was given a diagnosis of a suspicion of a prostatic cancer at the end of January 2003, when the inventor was staying in hospital of Japanese Red Cross Medical Center in Hiroo to undergo a complete medical checkup. According to a close examination during a stay in the hospital from February 8^{th} to 10^{th} in the same year, the marker value of serum blue brain examined in the blood drawing method on February 10^{th} in the same year was 7.5, though the normal value was 5. The inventor was thus diagnosed with prostatic cancer. According to a close examination on Jun. 23, 2003 in the same hospital, the marker value of serum blue brain had increased to 8.4.

At this time, there being no need for removing the prostate according to an opinion of a doctor, the inventor, who was a little concerned, tried moxa treatment. Although there was a prescribed medicine from the hospital, the inventor did not take the medicine in order to check the effectiveness of moxa treatment. Positions of moxa were determined for himself as follows, with reference to positions of pressure points: three points below the navel aligned in the vertical direction and a point on the perineum. Moxa treatment was done once a day.

Although there was a perceptible effect of moxa treatment, there being a need for troublesome handling of combustion ash of moxa, as described above in Background of invention, the present inventor developed the thermotherapy device of the above-described simplified embodiment. The device was brought into use as substitute for moxa from Jul. 22, 2003. The positions of heating were chosen as the same as in the case of moxa treatment, and the inventor used the device seeing how it works. Although there was a prescribed medicine from the hospital, the inventor did not take the medicine in order to check the effectiveness of the thermotherapy device.

According to a subsequent close examination on Sep. 29, 2003 at the same hospital, the marker value of serum blue brain had decreased to 7.9, and further, it had decreased to 6.8 according to another close examination on Jan. 61, 2004 at the same hospital. From Apr. 2 to Apr. 19, 2004, the inventor used the thermotherapy device of the embodiment to heat the skin to 50 to 60 degrees C. for two hours every day, an hour for each of the region below the navel and the perineum region. Consequently, the marker value of serum blue brain had decreased to 1.0 according to a close examination 18 days after that, on Apr. 20, 2004, at the same hospital. The prostatic cancer of the inventor of the present invention was judged to have been completely cured.

The reason for this may be presumed as follows:

in consideration of a theory that a cancerous cell is eliminated with a heat of 42 degree C. or more, with the use of the thermotherapy device of the present embodiment, since the heat radiation opening 1a having a diameter of 4 to 6 cm, and more preferably about 4.5 cm, prevents excessive concentration or diffusion of heat from the cartridge heater 2, and the cartridge heater 2, with the leading end thereof 2a located 1 to 3 cm, and more preferably about 2 cm, in the back of the heat radiation opening 1a, warms up the air inside the cup-like housing 1 in whole to transmit the heat onto the skin S by convection of the air as well as by infrared radiation from the leading end 2a, a substantial size of region R, including the surface and some centimeters backward thereof can be heated to a high temperature. By bringing the skin S to such a high temperature of 55 to 60 degrees C. as is sustainable for the patient, cancerous cells in the prostatic cancer are exposed as a whole to a sufficiently high temperature about 50 degrees C.

As described above, it is obvious that the thermotherapy device of the present embodiment is effective for treatment of cancer.

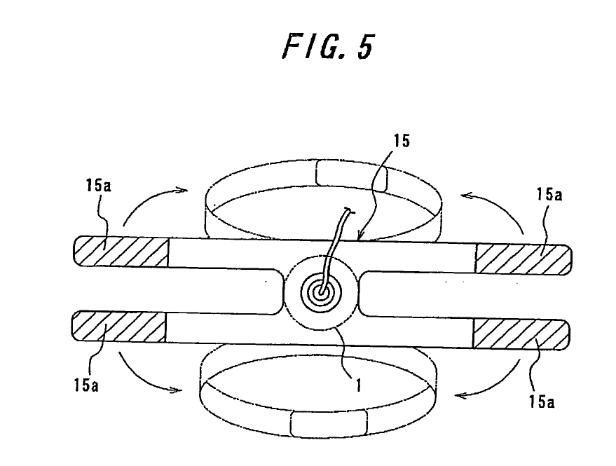

FIG. 5 is a plan view of the thermotherapy device according to another embodiment of the present invention. The thermotherapy device of the present embodiment differs from that of the first embodiment in that the device is provided with a belt 15 as a mounting fixture for fixing the heat radiation opening 1a of the housing 1 near the affected part. The both embodiments are the same in other respects.

The belt 15 is, for example, made of leather and substantially in the shape of H. The rear end of the housing 1 penetrates through a hole formed in the center of the belt to be fixed. The belt comprises on each of the four armlike portions thereof a hook and loop fastener (for example Magic Tape or Velcro Fastner™). The housing 1 can be fixed even on the perineum by folding each of the four armlike portions around the rootage of the legs of the patient, as shown by virtual lines in FIG. 5, and fastening the armlike portions into two loops with adjustment of length. Further, by folding the armlike portions around the trunk of the patient's body and fastening the armlike portions into two loops with adjustment of length, the housing may be fixed on the above-described region below the navel. Furthermore, the housing 1 may be fixed on the chest in the case of a breast cancer by fastening the armlike portions into the shape of X. The housing 1 may thus be provided with high fixability.

Accordingly, with the use of the thermotherapy device of the present embodiment, a burden on the patient can further be alleviated, since there is no need to hold the housing 1 by hand. Further, the housing can always be set on an accurate position near the affected part each time of repeated treatments, which will improve effectiveness of treatment.

Although the present invention has been described above with illustrated embodiments, the present invention is not limited thereto. The above-described controller, for example, may be provided with a timer for setting a start time, an ending time and a time period of power supply to the electric heater. The housing may also be made of heat resistance plastic or ceramics.

Further, the above-described mounting fixture is not limited to that of the above-described embodiment. The mounting fixture may comprise, as needed, a plurality of, more preferably three or more, armlike portions (belts) extending from the center where the housing is fixed, and the armlike portions are joined together integrally or in such a way that an angle between the armlike portions can be changed with the use of a hook and loop fastener or a snap or the like. In this case, after the armlike portions are folded about a leg, an arm or a trunk of the patient's body, the leading end of the armlike portions are fixed mutually or on the center with the hook and loop fasteners or the snaps or the like.

As a matter of course, the thermotherapy device of the present invention may be used effectively for treatment of other diseases than cancer.

### INDUSTRIAL APPLICABILITY

With the use of the thermotherapy device of the present invention, there is no need for replacement of a combustion rod or handling of combustion ash because a electric heater is used, and the device can be used even in bed. Further, due to the controller for supplying power to the electric heater with a control of the amount of heat generation, the skin may be heated to a high temperature of 55 to 60 degrees C. safely, bringing an affected part on or under the skin to such a high temperature as is effective and sufficient for treatment of diseases including cancer.

1. A thermotherapy device comprising:
a cup-like housing having a heat radiation opening;
a rod-like electric heater contained in said housing, the leading end thereof being directed toward said heat radiation opening; and
a controller for supplying power to said electric heater and controlling an amount of heat generation from said electric heater.

2. The thermotherapy device according to claim 1, wherein said heat radiation opening of the housing has a diameter in the range of 4 to 6 cm, and the leading end of said electric heater is located 1 to 3 cm in the back of the heat radiation opening.

3. The thermotherapy device according to claim 1, wherein a heat insulation material is provided between said housing and said electric heater.

4. The thermotherapy device according to claim 1, wherein a reflector is provided between said housing and said electric heater.

5. The thermotherapy device according to claim 1, wherein said housing is made of wood or ceramics.

6. The thermotherapy device according to claim 1, wherein a net member is provided on an inner side of said heat radiation opening of said housing.

7. The thermotherapy device according to claim 1, wherein a temperature sensor is provided near said heat radiation opening of said housing, and said controller controls an amount of heat generation from said electric heater, based on an output signal from said temperature sensor.

8. The thermotherapy device according to claim 1, wherein said controller is provided with a temperature adjustment knob for changing the amount of heat generation from said electric heater and a display area in which a correspondence between a turn position of the temperature adjustment knob and a target temperature is shown by different colors.

9. The thermotherapy device according to claim 1, further comprising a mounting fixture for fixing the heat radiation opening of said housing near a patient's affected part.

10. The thermotherapy device according to claim 2, further comprising a mounting fixture for fixing the heat radiation opening of said housing near a patient's affected part.

11. The thermotherapy device according to claim 3, further comprising a mounting fixture for fixing the heat radiation opening of said housing near a patient's affected part.

12. The thermotherapy device according to claim 4, further comprising a mounting fixture for fixing the heat radiation opening of said housing near a patient's affected part.

13. The thermotherapy device according to claim 5, further comprising a mounting fixture for fixing the heat radiation opening of said housing near a patient's affected part.

14. The thermotherapy device according to claim 6, further comprising a mounting fixture for fixing the heat radiation opening of said housing near a patient's affected part.

15. The thermotherapy device according to claim 7, further comprising a mounting fixture for fixing the heat radiation opening of said housing near a patient's affected part.

16. The thermotherapy device according to claim 8, further comprising a mounting fixture for fixing the heat radiation opening of said housing near a patient's affected part.
